(19) United States
(12) Patent Application Publication
(10) Pub. No.: US 2009/0074837 A1
(43) Pub. Date: Mar. 19, 2009

(54) Bioresorbable Calcium-Deficient Hydroxyapatite Hydrogel Composite

(75) Inventors: Barbara R. Evans, Oak Ridge, TN (US); Hugh M. O'Neill, Knoxville, TN (US); Stacy A. Hutchens, Knoxville, TN (US); Roberto Benson, Knoxville, TN (US)

Correspondence Address: UT-Battelle, LLC;Office of Intellectual Property, One Bethal Valley Road, 4500N, MS-6258, Oak Ridge, TN, 37831, US

(73) Assignee: UT-BATTELLE, LLC, Oak Ridge, TN (US)

(21) Appl. No.: 11/857,648

(22) Filed: Sep. 19, 2007

(51) Int. Cl. A61F 2/00; A61K 9/14; A61K 33/42; A61P 19/00

(52) U.S. Cl. 424/426; 424/484; 424/602

(57) ABSTRACT

The present invention provides a composite material comprising oxidized bacterial cellulose and calcium-deficient hydroxyapatite, and methods for preparing the composite material. The composite material is useful as a bone graft material. In another embodiment, the invention provides a method for tissue repair in a mammal. The method comprises inserting the composite material into cartilage or bone tissue.

The United States Government has rights in this invention pursuant to contract no. DE-AC05-00OR22725 between the United States Department of Energy and UT-Battelle, LLC.

### CROSS-REFERENCE TO RELATED APPLICATION

U.S. application Ser. No. 10/295,461 filed on Nov. 15, 2002 to Hutchens et al.

### FIELD OF THE INVENTION

The invention relates generally to the fields of biomaterials. More particularly, the invention relates to artificial bone compositions and methods of forming and using such compositions.

### BACKGROUND OF THE INVENTION

Cellulose, a polysaccharide, is a primary component of plant cell wall. Plant cellulose is generally associated with other biopolymers such as hemicellulose and lignin to form a laminate. Extracting cellulose from these other polymers requires harsh chemical processes, such as treatment with sulfur dioxide, sodium hydroxide and bleaching agents. This often causes the cellulose to lose strength.

Cellulose is currently used in medicine to produce, for example, dialysis membranes, drug coatings and blood coagulants. For use in medicine, cellulose is generally regenerated using the viscose process or is modified into derivatives such as cellulose acetate or carboxymethyl cellulose. These cellulose derivates have lost almost all of their crystallinity and native form.

Certain bacterial species possess the ability to secrete pure cellulose in the form of a hydrogel. When these bacteria are grown under appropriate conditions, they extrude microfibrils of pure highly crystalline cellulose. Bacterial cellulose is morphologically different from plant cellulose. The bacterial cellulose microfibrils are smaller with lateral dimensions one-half to one-tenth those of plant cellulose microfibrils. The crystalline microfibrils extruded by the bacteria as they move through the culture media form a sponge-like network in the bacterial growth media. In contrast, the cellulose microfibrils in plant cell walls are assembled into larger fibrous structures and are synthesized in association with other biopolymers that may include hemicellulose, lignin, waxes, and pectin, to form plant tissues. The bacterial cellulose fibrils are up to 200 times finer than cotton fibers, the purest form of native plant cellulose. These properties of bacterial cellulose thereby result in a large surface area. This permits a high density of inter- and intra-fibrillar hydrogen bonds which gives the cellulose its hydrogel structure.

Bacterial cellulose is a biocompatible material being investigated for a variety of medical applications, such as bone grafting. Bone grafting is a technique used to repair or help in the healing of osseous damage. The two most common methods used to restore bone are allografting and autografting.

Allografting involves the transplantation of tissue from a donor into a host subject. It has both clinical and practical drawbacks. For example, allografting exposes the host subject to a risk of acquiring an infection and/or disease and/or elicit a host immune system-mediated anti-graft response. In addition, donor tissue is often expensive or unavailable.

Autografting involves the transplantation of autologous tissue from one site in a subject's body to another site. The drawbacks of this procedure include limited supply of usable autologous bone. In addition, the procedure requires two surgical procedures (one to harvest the tissue, and one to transplant the tissue).

To avoid problems associated with allografting and autografting, synthetic bone-grafting materials have been developed. For example, the mineralized bacterial cellulose hydrogel composite material disclosed in U.S. Application Publication No. 2004/0096509 is a promising osseous biomaterial. However, humans lack the enzyme necessary to cleave the β-1,4 glycosidic bonds between glucose residues in the cellulose polymer. Thus, a major limitation of the hydrogel composite is the inability of the composite to degrade in mammalian systems.

Accordingly, it would be beneficial to provide a tissue substitute that is (i) biodegradable, (ii) bioabsorable, (iii) and resembles natural osseous tissue.

### SUMMARY OF THE INVENTION

These and other objectives have been met by the present invention, which provides, in one embodiment, a composite material comprising oxidized bacterial cellulose fibrils and calcium-deficient hydroxyapatite crystallites

In another embodiment, the invention provides a bone graft material comprising oxidized bacterial cellulose fibrils and calcium-deficient hydroxyapatite crystallites.

In yet another embodiment, the invention provides a method for preparing a composite material comprising oxidized bacterial cellulose fibrils and calcium-deficient hydroxyapatite crystallites. The method comprises incorporating calcium-deficient hydroxyapatite crystallites in a network of oxidized bacterial cellulose fibrils.

In a further embodiment, the invention provides a method for tissue repair in a mammal in need thereof. The method comprises providing a composite material comprising oxidized bacterial cellulose fibrils and calcium-deficient hydroxyapatite crystallites; and inserting said composite material into cartilage or bone tissue of the mammal, wherein said composite material supports cell colonization and is degradable and absorbable, thereby repairing tissue in the mammal

As a result of the present invention, an osseous tissue substitute that resembles natural osseous tissue is provided that is biodegradable, bioabsorable, and resembles natural osseous tissue.

For a better understanding of the present invention, together with other and further advantages, reference is made to the following detailed description, and its scope will be pointed out in the claims.

### BRIEF DESCRIPTION OF THE DRAWINGS

FIG. 1: (a) Wet weights and (b) dry weights of samples before in-vitro degradation.

FIG. 2: True-stress strain curve of (a) native BC, (b) oxidized BC, (c) native BC-CdHAP, and (d) oxidized BC-CdHAP.

FIG. 3: Comparison of (a) ultimate tensile strengths and (b) elastic moduli.

FIG. 4: X-ray diffraction patterns of (a) native BC, (b) oxidized BC, (c) native BC-CdHAP, and (d) oxidized BC-CdHAP.

FIG. 5: FTIR spectra of (a) oxidized BC-CdHAP, (b) oxidized BC, (c) native BC-CdHAP, and (d) native BC.

FIG. 6: SEM images of (a) native BC, (b) native BC-CdHAP, (c) oxidized BC, and (d) oxidized BC-CdHAP.

FIG. 7: Comparison of sample masses before and after incubation in HEPES buffer (pH 7.4) at 37° C. in static and dynamic conditions.

FIG. 8: Absorbance versus time of HEPES supernatant at (a) 240 nm and (b) 260 nm. ♦-oxidized BC, ▪-oxidized BC-CdHAP, -native BC-CdHAP, ▴-native BC.

### DETAILED DESCRIPTION OF THE INVENTION

The invention is based on the discovery by the inventors that a composite material comprising oxidized bacterial cellulose fibrils and calcium-deficient hydroxyapatite crystallites has the capacity to eventually be degraded and absorbed in the body of an animal and replaced by natural bone.

Throughout this specification, parameters are defined by maximum and minimum amounts. Each minimum amount can be combined with each maximum amount to define a range.

In one aspect, the invention provides a composite material comprising oxidized bacterial cellulose fibrils and calcium-deficient hydroxyapatite crystallites. Cellulose is a polysaccharide comprised of repeating glucose units. Cellulose formed by bacteria is referred to herein as bacterial cellulose fibrils. The cellulose produced by bacteria is usually pure, is highly crystalline and contains greater than 99% water. In addition, it forms an intricate highly interconnected porous network.

The bacterial cellulose useful in the composite materials of the present invention can be from any cellulose-producing bacteria. Examples of bacterial species that produce cellulose include, but is not limited to, bacteria from the genera of Aerobacter, Acetobacter (particularly Acetobacter species reclassified in 1997 as member of the genus Gluconoacetobacter, which genus is synonymous with the genus Gluconacetobacter, and is currently denoted by this genus name, Gluconacetobacter, by the American Type Culture Collection and the National Center for Biotechnology Information), Achromobacter, Agrobacterium, Alacaligenes, Azotobacter, Pseudomonas, Rhizobium, and Sarcina. Preferably, the bacteria species is Gluconoacetobacter xylinus or Gluconoacetobacter hansenii synonyms under which these species are known including Gluconacetobacter xylinus, Gluconacetobacter hansenii, Acetobacter xylinus, Acetobacter xylinum, and Acetobacter hansenii. Bacterial cellulose can be obtained commercially from food production companies in Southeast Asia or synthesized by bacteria. Methods for producing cellulose from bacteria are known to those in the art. See for example, the method disclosed in Example 1 below.

The bacterial cellulose useful in the composite materials of the present invention is oxidized. The term “oxidized bacterial cellulose fibrils” as used herein means that the hydroxyl (—OH) moieties at the C₂, C₃, or C₆ positions of glucose units in the bacterial cellulose fibrils are oxidized into aldehyde, carboxyl and/or ketone groups. The presence, however, of other oxidized groups in the fibrous material is not ruled out. In one embodiment, the oxidized bacterial cellulose is dialdehyde cellulose.

The cellulose chains of the bacterial cellulose can be partially or totally oxidized. The degree of oxidation of the bacterial cellulose fibrils is generally measured as a function of the amount of aldehyde, ketone, and/or carboxyl group content of the fibrous cellulose material. Usually, as the number of aldehyde, ketone, and/or carboxyl groups on the cellulose structure is increased, the oxidation content correspondingly increases.

The degree of oxidation of the bacterial cellulose fibrils will vary depending upon the time of the treatment and the relative proportion of the oxidizing agent to bacterial cellulose fibrils used in the manufacturing process. The oxidized bacterial cellulose fibrils used in the present invention has an aldehyde content of at least about 0.5 mol %, more preferably at least about 2 mol %, even more preferably at least about 5 mol % and most preferably at least about 10 mol %. The oxidized bacterial cellulose fibrils have an aldehyde content of at most about 90 mol %, preferably at most of at most about 85 mol %, even more preferably at most about 80 mol %, and most preferably at most about 75 mol %.

The oxidized bacterial cellulose can be produced by any method known to those skilled in the art. Typically, oxidized cellulose is produced by reacting cellulose with an oxidant. The oxidizing agents can be specific or unspecific in its action upon cellulose. Generally, oxidizing agents are unspecific in their action upon cellulose. Examples of suitable oxidants include gaseous chlorine, hydrogen peroxide, peracetic acid, chlorine dioxide, nitrogen dioxide (dinitrogen tetroxide), persulfates, permanganate, dichromate-sulfuric acid hypochlorous acid, hypochlorous acid, hypohalites or periodates. These oxidized celluloses may contain carboxylic, aldehyde, and/or ketone functionalities, in addition to the hydroxyl groups, depending on the nature of the oxidant and the reaction conditions used in their preparation.

Periodic acid and its salts are specific in their action upon cellulose in that they oxidize the vis-glycol groupings of the anhydro-D-glucose units to pairs of aldehyde groups. The periodates and nitrogen dioxide also differ from most other oxidizing agents in that they are able to penetrate and react with the crystalline as well as the amorphous portions of cellulose. With the common oxidizing agents, the reaction is typically confined to the amorphous regions and the surfaces of the cellulose crystallites. The oxidizing agents useful in the present invention are those agents that do not degrade the cellulose to the extent that the cellulose loses its fibrous form.

The composite materials of the present invention are degradable and absorbable in a physiological environment of a mammal. Degradation products of the oxidized bacterial cellulose fibrils include cellulooligosaccharides, small cellulose fragments, and carbonyl-containing molecules such as glycolic acid and 2,4-dihydroxybutyric acid. The degradation product can vary depending on the type of oxidized bacterial cellulose fibrils in the composite material. For example, the degradation products of dialdehyde cellulose include glycolic acid and 2,4-dihydroxybutyric acid. Such degradation products can safely be removed from the physiological system of a mammal by endogenous metabolic pathways, such as elimination by the kidneys or by entering the tricarboxylic acid cycle.

The composite material further contains hydroxyapatite. Hydroxyapatite, also known in the art as hydroxylapatite, is a mineral comprising calcium, phosphate and hydroxyl groups and has the chemical formula Ca₅(PO₄)₃(OH). Hydroxyapatite is commonly written as Ca₁₀(PO₄)₆(OH)₂ to denote that the crystal unit comprises two hydroxyapatite molecules. Ca₁₀(PO₄)₆(OH)₂ is referred to as the naturally occurring form of hydroxyapatite or stoichiometric hydroxyapatite. Stoichiometric hydroxyapatite has a calcium to phosphorous ratio of about 1.66 to about 1.67.

The hydroxyapatite useful in the composite materials of the present invention is not stoichiometric hydroxyapatite. Rather the hydroxyapatite useful in the present invention is deficient in calcium, and is referred to herein as “calcium-deficient hydroxyapatite.” Calcium-deficient hydroxyapatite has a calcium to phosphorus ratio of less than about 1.60. Preferably, the calcium-deficient hydroxyapatite has a calcium to phosphorus ratio in the range of about 1.33 to about 1.55. Calcium-deficient hydroxyapatite has the chemical formula shown in formula 1 below.

Ca₁₀₋ₓ(HPO₄)ₓ(PO₄)₆₋ₓ(OH)₂₋ₓ wherein 0<x≦1 Formula 1

In one embodiment, the hydroxyl ion can be replaced with a different ion. For example, the hydroxyl ion can be replaced with fluoride, chloride or carbonate.

The calcium-deficient hydroxyapatite in the composite material of the present invention is preferably in a crystalline form. The term “crystalline” as used herein means that the molecules of the calcium-deficient hydroxyapatite are packed in a regularly ordered, repeating pattern extending in all three spatial dimensions (a, b and c-axis). Calcium-deficient hydroxyapatite in crystalline form is referred to herein as calcium-deficient hydroxyapatite crystallites. In a preferred embodiment, the calcium-deficient hydroxyapatite crystallites are elongated in the c-axis, as compared to the a- and b-axis.

In one embodiment, the calcium-deficient hydroxyapatite crystallites are nanocrystallites. The term “nanocrystallites” as used herein refers to crystallites that are nanometer in size. The crystallites can be any nanometer in size, such as for example, 10-50 nm.

In one embodiment, the oxidized bacterial fibril network is impregnated with calcium-deficient hydroxyapatite crystallites. For example, the oxidized cellulose fibril network can be soaked in a solution containing calcium-deficient hydroxyapatite crystallites.

Alternatively, the calcium-deficient hydroxyapatite crystallites are chemically bonded to oxidized bacterial cellulose fibrils. The bond can be, for example, a hydrogen bond, ionic bond, covalent bond, non-covalent bond, electrostatic bond, London's force, and van der Waals force.

The calcium-deficient hydroxyapatite can be chemically bonded to any functional group on the oxidized bacterial cellulose fibrils. In a preferred embodiment, the calcium-deficient hydroxyapatite is chemically bonded to aldehyde groups and/or hydroxyl groups on the oxidized bacterial cellulose fibrils. For example, a chemical bond can be formed between aldehyde groups or hydroxyl groups on the oxidized bacterial cellulose fibrils and calcium cations on the calcium-deficient hydroxyapatite. Suitable methods for preparing composite material containing oxidized bacterial fibrils chemically bonded to calcium-deficient hydroxyapatite crystallites are described below.

The composite material of the present invention can contain any amount of calcium-deficient hydroxyapatite. Generally, the composite material contains at least about 5% calcium-deficient hydroxyapatite, generally at least about 10% calcium-deficient hydroxyapatite, even more generally at least about 15% calcium-deficient hydroxyapatite, and most generally at least about 20% calcium-deficient hydroxyapatite. Typically, the composite materials contains at most about 95% calcium-deficient hydroxyapatite, more typically at most about 90% calcium-deficient hydroxyapatite, even more typically at most about 85% calcium-deficient hydroxyapatite, and most typically at most about 80% calcium-deficient hydroxyapatite.

The calcium-deficient hydroxyapatite produced in oxidized bacterial cellulose is similar in morphology and chemistry to that of natural bone apatite. In a preferred embodiment, the calcium-deficient hydroxyapatite is degradable and can be absorbed by a mammal.

The composite material is in the form of a hydrogel when exposed to water. The term “hydrogel” (also known as aquagel) as used herein refers to superabsorbent polymeric networks that can retain large volumes of water in their swollen structures. The composite materials of the present invention maintain their three-dimensional fibril networks when in the hydrogel form.

The composite material can be packaged and stored in a variety of ways. For example, the composite material can be stored and maintained in a hydrated state, such as a hydrogel, for an extended period of time. Alternatively, the composite material can be freeze-dried and stored in an essentially desiccated state until use.

In one embodiment, one or more bio-active agents can be incorporated into the composite materials of the present invention. The term “bio-active agent” as used herein is an agent that exerts a biological effect. The bio-active agent can be released via diffusion. For example, the bio-active agent can be dissolved in a solvent and loaded into the composite material. Alternatively, the bio-active agent can be bound, via surface adsorption, to the bacterial cellulose fibrils and/or calcium-deficient hydroxyapatite crystallites. In such cases, degradation of the composite material is not necessary for release of the bio-active agent, since the bio-active agent would typically be released upon implantation. Conversely, the bio-agent agent can be released in a controlled manner as the composite material is degraded and absorbed. For example, such composite materials can be engineered to degrade and be absorbed at particular rates by selecting, for instance, the amount of calcium-deficient hydroxyapatite crystallites in the composite material, the particular type of oxidized bacterial cellulose fibrils employed, the degree of oxidization of the oxidized cellulose fibrils, etc. The bio-active agent could also be chemically attached to the cellulose molecule and its subsequent release would be dependent on the type of chemical linkage.

Any bio-active agent can be incorporated into the composite material of the present invention provided that the agent(s) does not interfere with the required characteristics and functions of the composite material. Preferably, the bio-active agents promote bone regeneration and/or growth or prevent infection.

The bio-active agent can be a biological molecule, a small molecule, a macromolecule, or a therapeutic agent. Biological molecules include all lipids and polymers of monosaccharides, amino acids and nucleotides typically having a molecular weight greater than 450 Da. Thus, biological molecules include, for example, oligosaccharides and polysaccharides; oligopeptides, polypeptides, peptides, and proteins; and oligonucleotides and polynucleotides.

Biological molecules further include derivatives of any of the molecules described above. For example, derivatives of biological molecules include lipid and glycosylation derivatives of oligopeptides, polypeptides, peptides and proteins. Derivatives of biological molecules further include lipid and glycosylated derivatives of oligosaccharides and polysaccharides, e.g. lipopolysaccharides. Examples of biological molecules include, but are not limited to, bone morphogenetic proteins (e.g., BMP1, BMP2, BMP3, BMP4, BMP5, BMP6, BMP7), prostaglandins, alkaline phosphatase, glycosaminoglycans, osteocalcin, osteonectin, bone sialo proteins, epidermal growth factor, fibroblast growth factor, platelet derived growth factor, transforming growth factor, vascular endothelial growth factor.

Any molecule that is not a biological molecule is considered in this specification to be a small molecule or a macromolecule. Accordingly, small molecules and macromolecules include organic compounds, organometallic compounds, salts of organic and organometallic compounds, saccharides, amino acids, nucleotides, and ions (e.g., sodium, fluoride, chloride, etc.). Small molecules further include molecules that would otherwise be considered biological molecules, except their molecular weight is not greater than 450 Da. Thus, small molecules may be lipids, oligosaccharides, oligopeptides, and oligonucleotides, and their derivatives, having a molecular weight of 450 or less. Similarly, biological molecules that have their molecular weights greater than 450 Da can also be referred to herein as macromolecules.

It is emphasized that small molecules can have any molecular weight. They are merely called small molecules because they typically have molecular weights less than 450. Small molecules and macromolecules include compounds that are found in nature as well as synthetic compounds.

Therapeutic agents are typically medically useful drugs. The therapeutic agent can be a biological molecule, a small molecule or a macromolecule. Examples of therapeutic agents include, for example, without limitation, thrombo-resistant agents, antibiotic agents, anti-tumor agents, anti-viral agents, anti-angiogenic agents, angiogenic agents, anti-inflammatory agents, cell cycle regulating agents, their homologs, derivatives, fragments, pharmaceutical salts and combinations thereof.

Useful thrombo-resistant agents can include, for example, heparin, heparin sulfate, hirudin, chondroitin sulfate, dermatan sulfate, keratin sulfate, lytic agents, including urokinase and streptokinase, their homologs, analogs, fragments, derivatives and pharmaceutical salts thereof.

Useful antibiotics can include, for example, penicillins, cephalosporins, vancomycins, aminoglycosides, quinolones, polymyxins, erythromycins, tetracyclines, chloramphenicols, clindamycins, lincomycins, sulfonamides, their homologs, analogs, fragments, derivatives, pharmaceutical salts and mixtures thereof.

In another embodiment, the composite material can further be seeded with cells. Preferably, the cells are those that are useful for promoting bone growth and/or regeneration. Examples of such cells include, but are not limited to, osteoprogenitor cells, mesenchymal stem cells, chondrocytes, osteoblasts, osteocytes, and fibroblasts.

In another aspect, the invention provides a method for preparing a composite material comprising oxidized bacterial cellulose fibrils and calcium-deficient hydroxyapatite crystallites. The first step in the method is to provide a network of oxidized bacterial cellulose fibrils.

The next step in the method for preparing the composite material of the present invention is to incorporate calcium-deficient hydroxyapatite crystallites into the network of oxidized bacterial cellulose fibrils.

Any method can be utilized to incorporate the calcium-deficient hydroxyapatite crystallites into the network of oxidized bacterial cellulose fibrils. In one embodiment, the calcium-deficient hydroxyapatite crystallites can be incorporated into the network of oxidized bacterial cellulose fibrils by incubating the network of oxidized bacterial cellulose fibrils in a solution containing calcium-deficient hydroxyapatite crystallites. The incubation conditions will vary depending on various parameters, such as the temperature of incubation, the time length of incubation, the concentration of the calcium-deficient hydroxyapatite crystallites solution, pH of the solution, the size of the calcium-deficient hydroxyapatite crystallites, and the amount of calcium-deficient hydroxyapatite crystallites desired in the network of oxidized bacterial cellulose fibrils.

Generally, the concentration of the calcium-deficient hydroxyapatite crystallites is, but not limited to, about 0.1-20%.

In another embodiment, the calcium-deficient hydroxyapatite crystallites can be incorporated into the network of oxidized bacterial cellulose fibrils by incubating the network of oxidized bacterial cellulose fibrils in a calcium salt solution and a phosphate salt solution. Incubation of the network of oxidized bacterial cellulose fibrils with a calcium salt solution and a phosphate salt solution can occur simultaneously or sequentially.

For example, the network of oxidized bacterial cellulose fibrils can be incubated in a calcium salt solution followed by incubation in a phosphate salt solution. Alternatively, the network of oxidized bacterial cellulose fibrils can be incubated in a phosphate salt solution followed by incubation in a calcium salt solution. The incubation in the salt solution is generally carried out at least one or more times, and depends on various parameters such as, the concentration of the calcium salt solution, the concentrations of the phosphate salt solution, the size of the calcium-deficient hydroxyapatite crystallites desired, and the amount of calcium-deficient hydroxyapatite crystallites desired in the network of oxidized bacterial cellulose fibrils.

Any calcium salt solution can be employed. Examples of suitable calcium salt solutions include, but are not limited to, calcium chloride, calcium phosphate, calcium nitrate, calcium carbonate, calcium citrate, calcium malate, calcium gluconate, calcium oxide, and combination thereof. Generally, the concentration of the calcium salt solution is, but not limited to, about 5 mM-2 M.

Any phosphate salt solution can be employed. Examples of suitable phosphate salt solutions include, but are not limited to, sodium phosphate dibasic, potassium phosphate dibasic, sodium tripolyphosphate, diammonium phosphate, and combinations thereof. Generally, the concentration of the phosphate salt solution is, but not limited to, about 5 mM-2 M.

In yet another embodiment, the calcium-deficient hydroxyapatite crystallites can be incorporated into the network of oxidized bacterial cellulose fibrils by incubating the network of oxidized bacterial cellulose fibrils in simulated body fluid. The term “simulated body fluid” as used herein refers to any solution that mimics a body fluid. Preferably, the simulated body fluid has an ion concentration similar to that of blood plasma.

In yet a further embodiment, the network of oxidized bacterial cellulose fibrils is phosphorlyated. Thus, in this embodiment, the calcium-deficient hydroxyapatite crystallites can be incorporated into the network of phosphorylated oxidized bacterial cellulose fibrils by incubating it in a calcium salt solution. Examples of suitable calcium salt solutions include those described above. Incubation of the phosphorylated oxidized bacterial cellulose fibrils in a calcium salt solution results in formation of calcium-deficient hydroxyapatite crystallites that are chemically bound to the oxidized bacterial cellulose fibrils.

The optimization of incubation conditions for a composite material having a given property (e.g., weight percent of calcium-deficient hydroxyapatite crystallites) is within the skill of those in the art.

Due to the ability of the composite materials of the present invention to be degraded and absorbed, the composite materials can be used to regenerate bone or cartilage when implanted into osseous defects of a mammal. Thus, in yet another aspect of the invention, the composite material is a synthetic bone graft material.

In a further aspect of the invention, the composite material described above can be used as a substitute or scaffold in tissue engineering. Thus, in another aspect, the present invention provides a method for tissue repair in a mammal in need thereof. Any mammal may be treated in accordance with the invention. Mammals include, for example, humans, baboons and other primates, as well as pet animals such as dogs and cats, laboratory animals such as rats and mice, and farm animals such as horses, sheep, and cows. Preferably, the mammal is a human.

Mammals in need of tissue repair include mammals that have damaged or injured their cartilage or bone tissue. The method comprises providing the composite material comprising oxidized bacterial cellulose fibrils and calcium-deficient hydroxyapatite crystallites described above, and inserting the composite material into the cartilage or bone tissue of the mammal. The calcium-deficient hydroxyapatite in the composite material promotes bone regeneration and growth and cell colonization, thereby repairing tissue (e.g., osseous defects) in the mammal.

The composite material is not limited to its use as a synthetic bone grafting material. Other applications for the composite material will be apparent to those skilled in the art. For example, the composite material can also be used for absorption of materials, such as the absorption of proteins. In addition, the composite material can be used as a source for binding water or soil contaminants such as lead, cadmium, zinc, uranium, strontium, and etc. Further, the composite material can be used as a chromatography medium, for example, to remove metal ions from aqueous solutions.

### EXAMPLES

### Example 1

### Materials and Methods

Synthesis of bacterial cellulose. The bacterial strain Gluconacetobacter hansenii was obtained from American Type Culture Collection (Manassas, Va., U.S.A.) (ATCC 10821). Cellulose was synthesized by an optimized modification (Hutchens et al., Lett. Appl. Microbiol., 2007, 44:175-180) of the method of Schramm and Hestrin (J. Gen. Microbiol., 1954, 11: 123-129). The pellicle discs of cellulose (6 cm diameter, ˜3 mm thickness) synthesized by the bacteria, were harvested following culture for thirty days and purified.

Bacterial cellulose oxidation. Twenty-two cellulose pellicles were placed in a capped vessel containing 145 ml of 50 mM NaIO₄ in 5% n-propanol. The vessel was covered in aluminum foil and placed on an orbital shaker for 24 h at 23° C. The reaction was stopped by placing the vessel in an ice bath and adding 0.5 ml of glycerol to consume the excess periodate. The cellulose was then purified with several changes of distilled/deionized water. The remaining 22 unmodified cellulose pellicles were stored in distilled/deionized water.

Deposition of calcium-deficient hydroxyapatite. Calcium-deficient hydroxyapatite (CdHAP) was formed in the native and oxidized bacterial cellulose (BC) by performing alternating incubation cycles with calcium and phosphate solutions. Eleven native BC pellicles and eleven oxidized BC pellicles were each placed in a vessel containing 250 ml of 100 mM CaCl₂. The vessels were placed on an orbital shaker for 24 h at 23° C. The pellicles were rinsed in distilled/deionized water, then each set was placed in a vessel containing 250 ml of 60 mM Na₂HPO₄ and subjected to agitation for another 24 h (23° C.). The cellulose was then rinsed and stored in distilled/deionized water.

Four sets of 11 samples were made in all: native BC, native BC-CdHAP, oxidized BC, and oxidized BC-CdHAP. After synthesis, the wet weights of three samples from each group were determined. The samples were then dried and re-weighed to determine the dry weight. JMP software (SAS: Cary, N.C., U.S.A.) was used to determine statistically significant weight differences by performing a Tukey-Kramer test at α=0.05. The amounts of CdHAP in the composites were calculated by subtracting the total mineralized cellulose weight from the average weight of the unmineralized cellulose weight. CdHAP weight percentages were calculated by dividing the mass of the CdHAP by the total composite weight.

Material characterization. To determine the mechanical properties, hydrated samples were cut into strips (˜40 mm×20 mm×3 mm) and mounted onto a universal testing machine (Model SFM-20 United Calibration Corporation: Huntington Beach, Calif., U.S.A.) with hydraulic clamps. A 10 lb load cell was used to extend the samples from a 0.5 inch gauge length with a crosshead speed of 1 inch/minute until failure. JMP software was used to determine statistical significance by performing a Tukey-Kramer test at α=0.05.

X-ray diffraction was performed on dried samples mounted on a silicon zero-background holder. Data were obtained on a X'Pert PRO MPD X-Ray Diffractometer (PANalytical: Almelo, Netherlands) operated at 45 kV and 40 mA with CuKα radiation. Data were collected between 10 and 70° 2θ at a scan rate of 1° 2θ/min. Jade® 6.0 Software (Materials Data Incorporated: Livermore, Calif., U.S.A.) was used to identify the crystal structure of the cellulose and calcium-deficient hydroxyapatite.

Dried samples were measured on a BioRad FTS6000 Fourier Transform Infrared Spectrometer (BioRad: Randolph, Mass., U.S.A.). The spectra are the result of 256 scans collected with a resolution of 4 cm⁻¹.

Lyophilized samples were mounted on carbon tape and sputtered with gold on a Spi Module Sputter Coater (Spi Supplies: Westchester, Pa., U.S.A.) at 20 mA for 10 s. They were then analyzed on a LEO 1525 Scanning Electron Microscope (Zeiss: Oberkochen, Germany). The native BC and oxidized BC-CdHAP were imaged with a 3 kV accelerating voltage, and the oxidized BC and native BC-CdHAP were imaged at 5 kV.

In-vitro degradation testing Eight samples of each group were placed in HEPES buffer (25 mM HEPES, 75 mM NH₄Cl, pH 7.4) and autoclaved at 121° C. for 20 minutes. Each of the samples were then placed in a sterile 50 ml conical tube and added to 5 ml of sterile HEPES buffer. Four of the samples of each group were placed in a 37° C. Form a Scientific Water Jacketed Incubator (Thermo Electron Corporation: Waltham, Mass., U.S.A.) for static incubation. The remaining four samples of each group were placed in a 37° C. controlled environment orbital shaking incubator (New Brunswick Scientific Co., Inc.: Edison, N.J., U.S.A.) for dynamic incubation.

Every 48 h, the samples were separated from the HEPES buffer by centrifugation at 3500×g for 15 min. The absorbance of the supernatant was read on a Cary spectrophotometer (Varian: Palo Alto, Calif., U.S.A.) from 190 nm-350 nm. Fresh sterile HEPES buffer (5 ml) was then added to each sample of cellulose before being returned to the 37° C. incubators. The samples were removed from the HEPES buffer after fourteen days of incubation, rinsed in several changes of distilled/deionized water, dried, and weighed.

Identification of degradation products. A second batch of samples was made in a similar manner to produce a set of native BC, oxidized BC, native BC-CdHAP, and oxidized BC-CdHAP. Each set had six samples and were autoclaved in HEPES buffer. Like the previous study, each sample was placed in a sterile conical tube and 5 ml of sterile HEPES buffer was added. Three of the six samples of each set were placed statically incubated at 37° C., while the remaining three samples of each group were placed in a 37° C. shaking incubator. Unlike the previous study, the samples remained in the original HEPES buffer for the entire 14 day period. At the end, the samples were separated from the HEPES buffer by centrifugation at 3500×g for 15 min. The supernatant was then assayed for free carbohydrate.

To determine the amount of free carbohydrate, 0.5 ml of the supernatant was added to 2 ml of 0.16% anthrone (Fisher Scientific: Waltham, Mass., U.S.A.) in concentrated sulfuric acid. Standard solutions (0.5 ml volume) with concentrations ranging from 5-100 μg/ml glucose in low salt HEPES buffer were also added to 2 ml of the anthrone solution. The mixtures were vortexed, incubated at 90° C. for 20 min, then allowed to cool. The optical densities of the solutions were read at 587 nm using HEPES buffer as a blank. The concentrations were determined from a standard curve.

### Example 2

### Characterization of Native and Oxidized Bacterial Cellulose Calcium-Deficient Hydroxyapatite Composites

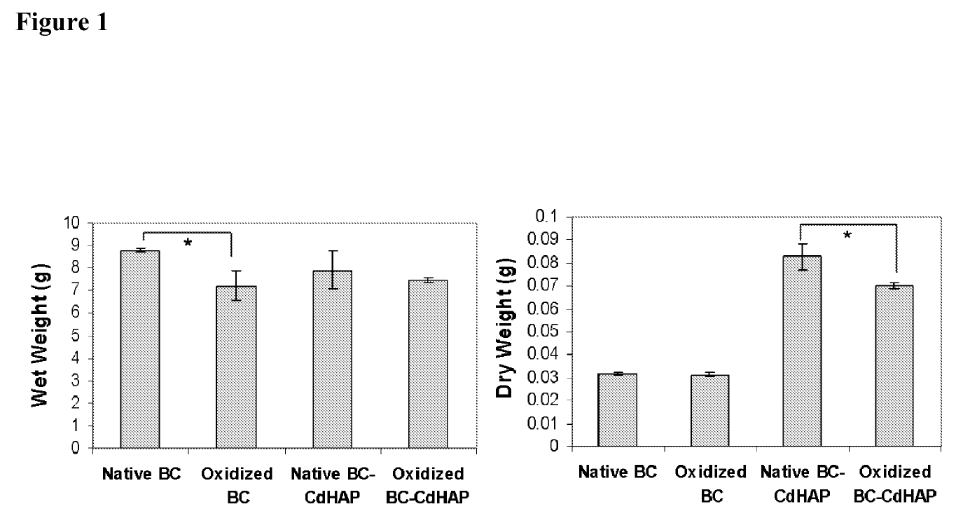

The wet weight of the oxidized cellulose samples was significantly less than the native cellulose samples (FIG. 1a), although the dry weights were not significantly different (FIG. 1b). Thus, during periodate oxidation, a change in the cellulose network structure occurs which decreases its capacity to retain water.

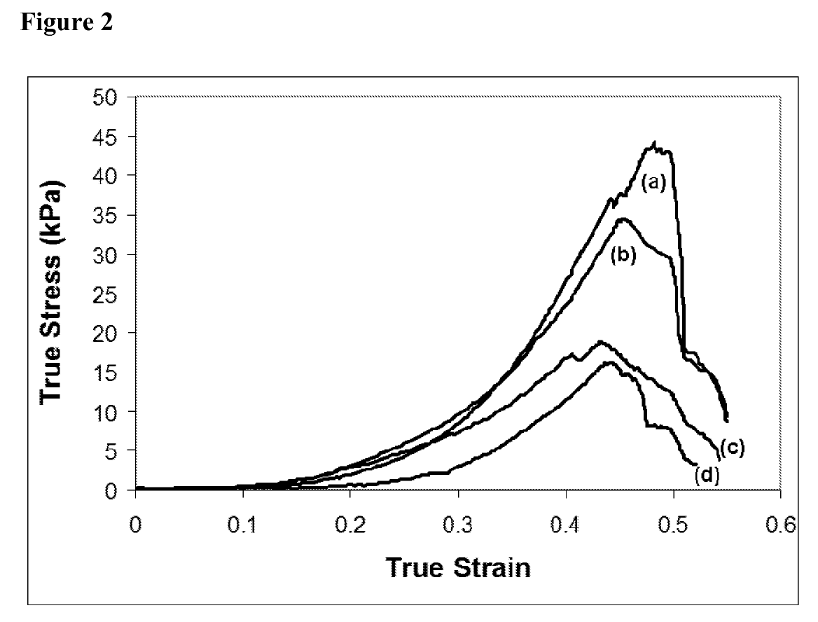

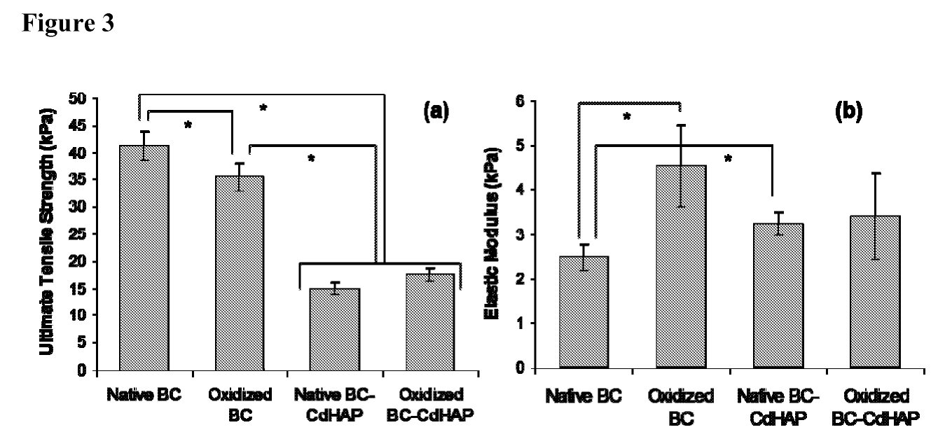

A homogenous white precipitate immediately formed throughout the bacterial cellulose (BC) matrix when it was placed in a phosphate solution after incubation in aqueous calcium chloride. After drying and weighing the samples, it was observed that more calcium-deficient hydroxyapatite (CdHAP) formed in the native BC compared to the oxidized BC (FIG. 1b) (62% CdHAP in native composite vs. 55% CdHAP in oxidized composite). The true stress-strain curves of the hydrated samples are shown in FIG. 2. The ultimate tensile strength and elastic modulus values of the samples are compared in FIG. 3. After oxidation and mineralization, the samples lose strength and become stiffer.

The altered network structure and hydrogen bonding accounts for the weaker strength of oxidized BC (FIG. 3). In addition, formation of the CdHAP crystallites which are bonded to the cellulose hydroxyl groups interrupts the natural hydrogen bonding causing weaker mechanical properties. It was also seen that more calcium-deficient hydroxyapatite forms in native cellulose than in oxidized cellulose. The native structure and location of the hydroxyl groups in unmodified bacterial molecule may be more conducive for apatite nucleation versus the aldehyde groups created in the oxidized cellulose.

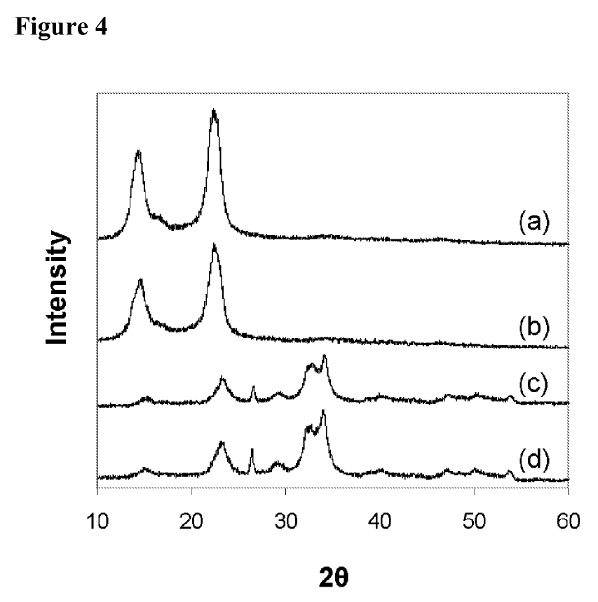

The x-ray diffraction patterns of the cellulose samples are presented in FIG. 4. The x-ray diffraction (XRD) pattern of native cellulose (FIG. 4a) showed the crystalline structure of BC as given by sharp defined peaks (PDF#03-0289). Periodate oxidation did not appreciably affect the crystalline structure of bacterial cellulose (FIG. 4b).

Likewise, the crystal structure is identified as calcium-deficient hydroxyapatite in the oxidized BC-CdHAP sample (PDF#46-0905). This confirms that the oxidized cellulose retained its ability to form CdHAP (FIG. 4d).

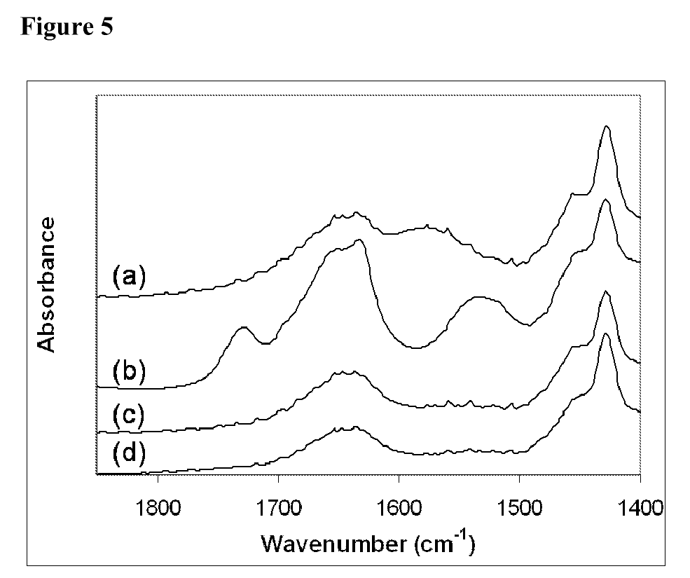

The FTIR spectra of the native and oxidized cellulose samples are shown in FIG. 5. In the oxidized BC spectrum, the absorption band at 1740 cm⁻¹ corresponds to the aldehyde carbonyl group and the band at 1652 cm⁻¹ represents carbonyl groups involved in hydrogen bonding (FIG. 5b). The native BC and native BC-CdHAP IR spectra are devoid of these absorption bands (FIGS. 5c and 5d). After depositing CdHAP into the oxidized BC, the carbonyl absorption bands shift to lower wavenumbers (FIG. 5a).

FTIR confirmed that the cellulose oxidized into dialdehyde cellulose after treatment with sodium periodate. Both carbonyl absorption bands shifted to lower wavenumbers after deposition of CdHAP indicating that a chemical bond had formed. The data shows that a chemical bond had formed between the negative dipole of the cellulose aldehyde groups and the calcium cation of the CdHAP mineral. Having the CdHAP particles chemically bound to the oxidized BC is beneficial since the composite will stay conjunct upon implantation. In addition, the CdHAP particles are prevented from being released which could cause damage to the surrounding tissues and initiate dystrophic calcification.

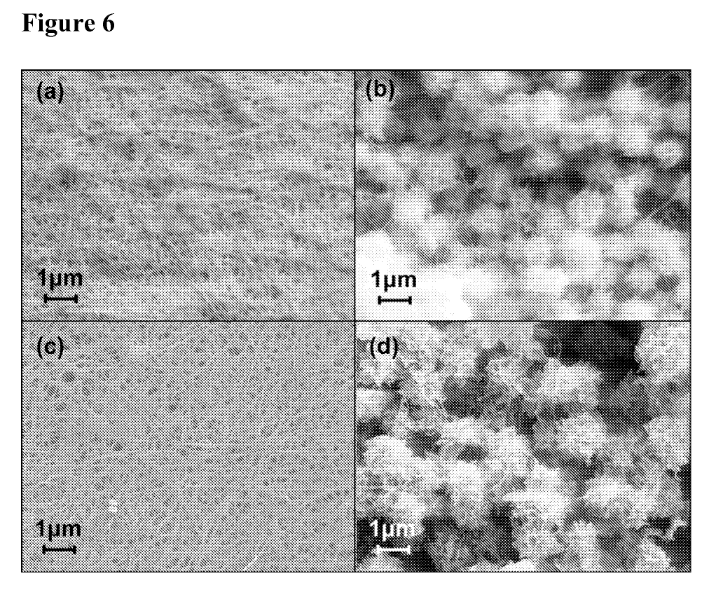

SEM images of the samples are shown in FIG. 6. The fine fibrils of native BC are seen in FIG. 6a. The oxidized BC is also composed of fine fibrils (FIG. 6b). In the native BC-CdHAP sample, it is observed that regular 1 μm spherical hydroxyapatite clusters had formed within the cellulose fibrils composed of discrete needle and plate-like crystallites (FIG. 6c). The oxidized BC-CdHAP sample also contained 1 μm clusters with a similar morphology (FIG. 6d).

### Example 3

### Degradation of Oxidized Bacterial Cellulose

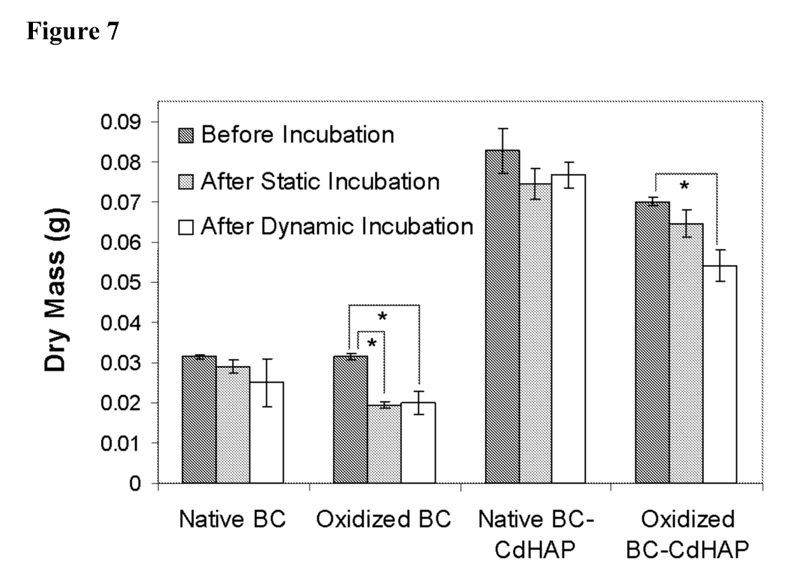

The dry weights of the samples before and after the in-vitro degradation period are given in FIG. 7. Statistical analysis showed that oxidized BC lost significant mass after static and dynamic incubation in the HEPES buffer at 37° C. (an average decrease of 38% and 36% respectively). However, the oxidized BC weight losses after static and dynamic incubation were not significantly different. This shows that degradation of periodate oxidized bacterial cellulose is not attributed to physical disruption, but is a result of hydrolysis in an aqueous environment at physiological pH. Oxidized BC-CdHAP lost significant weight after dynamic incubation in the HEPES buffer at 37° C., losing 23% of its mass on average. Mechanical disruption therefore did affect the mass loss of the oxidized BC-CdHAP composite. The chemical bonding between the oxidized BC and CdHAP may not be as strong as that between the native BC and CdHAP, causing some of the CdHAP particles to detach from the oxidized BC when it is continuously agitated.

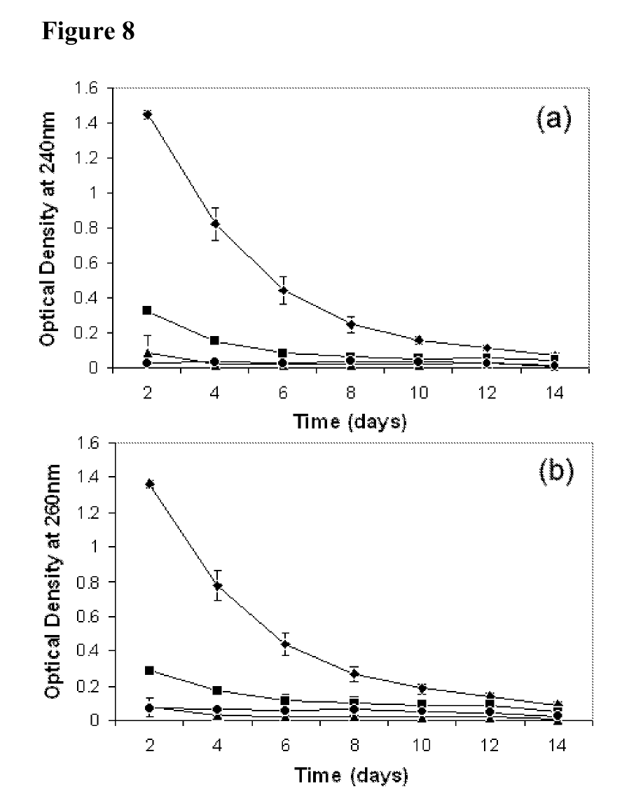

The degradation behavior of the samples in low-salt HEPES buffer was determined by following the changes in the absorbance of the supernatant. Absorption bands at 240 nm and 260 nm were observed in the oxidized BC and oxidized BC-CdHAP samples. The absorbance versus time plots for the static samples at 240 nm and 260 nm are given in FIG. 8. The graphs of the dynamic samples had similar results. It was seen that oxidized BC released a high concentration of degradation products that absorbed at 240 nm and 260 nm during the early time periods which eventually decayed to an equilibrium value. The oxidized BC-CdHAP samples also released degradation products in a similar manner, though not as much as the oxidized BC alone. Native BC and native BC-CdHAP did not release a significant amount of degradation products that absorbed at 240 and 260 nm.

### Example 4

The calorimetric assay with anthrone in concentrated sulfuric acid showed that all of the samples released free carbohydrate into the HEPES buffer. This indicates that both oxidized and native BC breaks down into smaller cellulose molecules when placed in a physiological environment. Variance was too high between the results to determine whether certain samples released statistically significantly more carbohydrate than others.

### Example 5

The calcium-deficient hydroxyapatite (CdHAP) crystallites in the composite material are 10-50 nm in size. Thus, the composite material is smooth and nonabrasive and prevents damage to neighboring soft tissues. Also, since the CdHAP is immobilized in the oxidized cellulose membrane, the particles remain in the defect site and do not migrate to other areas when implanted. Due to these advantages, the composite material of the present invention can be used in bone grafting procedures such as site extraction preservation and sinus lifts.

A mammal is anesthetized and the composite material, which is a soft pliable membrane which can easily be packed into a variety of defect sizes and shapes, is implanted in an osseous defect in the jaw. Implantation of the composite material results in regeneration of bone in the jaw in close proximity to soft tissues, including the gingiva and sinus membranes.

Thus, while there have been described what are presently believed to be the preferred embodiments of the invention, changes and modifications can be made to the invention and other embodiments will be know to those skilled in the art, which fall within the spirit of the invention, and it is intended to include all such other changes and modifications and embodiments as come within the scope of the claims as set forth herein below.

What is claimed is:

1. A composite material comprising oxidized bacterial cellulose fibrils and calcium-deficient hydroxyapatite crystallites.

2. The composite according to claim 1, wherein the oxidized bacterial cellulose is degradable when implanted in a mammal.

3. The composite according to claim 1, wherein the oxidized bacterial cellulose is absorbable.

4. The composite according to claim 2, wherein the degradation products of oxidized bacterial cellulose is glycolic acid and 2,4-dihydroxybutryic acid.

5. The composite according to claim 1, wherein the oxidized bacterial cellulose is dialdehyde cellulose.

6. The composite according to claim 1, wherein the calcium-deficient hydroxyapatite crystallites are chemically bonded to the oxidized bacterial cellulose fibrils.

7. The composite according to claim 6, wherein the calcium-deficient hydroxyapatite is attached to functional groups present on the oxidized bacterial cellulose fibrils.

8. The composite according to claim 7, wherein the functional groups are aldehyde groups and hydroxyl groups on the oxidized bacterial cellulose fibrils.

8. The composite according to claim 6, wherein the chemical bond is formed between aldehyde groups and hydroxyl groups on the oxidized bacterial cellulose fibrils and calcium cations on the calcium-deficient hydroxyapatite crystallites.

10. The composite according to claim 1, wherein the calcium-deficient hydroxyapatite is degradable when implanted in a mammal.

11. The composite according to claim 1, wherein the calcium-deficient hydroxyapatite is absorbable when implanted in a mammal.

12. A bone graft material comprising oxidized bacterial cellulose fibrils and calcium-deficient hydroxyapatite crystallites.

13. The bone graft material according to claim 12, wherein the calcium-deficient hydroxyapatite crystallites are chemically bonded to the cellulose fibrils.

14. A method for preparing a composite material comprising oxidized bacterial cellulose fibrils and calcium-deficient hydroxyapatite crystallites, the method comprising incorporating calcium-deficient hydroxyapatite crystallites in a network of oxidized bacterial cellulose fibrils.

15. The method according to claim 14, wherein the network of oxidized bacterial cellulose fibrils is incubated in a calcium-deficient hydroxyapatite crystallite solution.

16. The method according to claim 14, wherein the network of oxidized bacterial cellulose fibrils is incubated in a calcium salt solution and a phosphate salt solution.

17. The method according to claim 16, wherein the incubation in the calcium salt solution and the phosphate salt solution is done simultaneously.

18. The method according to claim 16, wherein the incubation in the calcium salt solution and the phosphate salt solution is done sequentially.

19. The method according to claim 14, wherein the oxidized bacterial cellulose fibrils is phosphorylated oxidized bacterial cellulose fibrils.

20. The method according to claim 14, wherein the phosphorylated oxidized bacterial cellulose fibrils is incubated in a calcium salt solution.

21. The method according to claim 14, wherein the network of oxidized bacterial cellulose fibrils is incubated in a simulated body fluid.

22. A method for tissue repair in a mammal in need thereof, the method comprising:
(i) providing a composite material comprising oxidized bacterial cellulose fibrils and calcium-deficient hydroxyapatite crystallites; and
(ii) inserting said composite material into cartilage or bone tissue of the mammal
wherein said composite material supports cell colonization and is degradable and absorbable, thereby repairing tissue in the mammal.
